(19) United States
(12) Patent Application Publication
(10) Pub. No.: US 2024/0285862 A1
(43) Pub. Date: Aug. 29, 2024

(54) Autoinjector

(71) Applicant: Becton Dickinson France, Le Pont-de-Claix (FR)

(72) Inventors: Michael Fiard, Eybens (FR); Nicolas Besson, Treffort (FR); Rémi Jamon, Vaulnaveys le Haut (FR); Pierre Ducarouge, Grenoble (FR); Louis Petit, Venon (FR)

(21) Appl. No.: 18/572,879

(22) PCT Filed: Jun. 21, 2022
(86) PCT No.: PCT/EP2022/066907

§ 371 Date: Dec. 21, 2023

(30) Foreign Application Priority Data
Jun. 25, 2021 (EP) 21305880.3

(51) Int. Cl. A61M 5/315; A61M 5/32

(52) U.S. Cl. CPC A61M 5/315; A61M 5/3243

(57) ABSTRACT

The autoinjector includes a housing configured to receive a medical container having a barrel defining a reservoir for containing a medical product, and said barrel having a distal end provided with a needle and an opened proximal end configured to receive a plunger rod for pushing a stopper arranged inside the barrel. The autoinjector also includes a needle cover coupled to and movable with respect to said housing between a first extended position, a retracted position, and a second extended position, an injection mechanism configured to move the plunger rod distally inside the barrel in order to expel the medical product contained inside the barrel, and a holder movable with respect to the housing between a passive position wherein the holder does not trigger the injection mechanism and an active position wherein the holder triggers the injection mechanism. A locker is coupled to the holder and is movable with regard to the housing between a locking position, thereby preventing the holder from moving to the activated position, and an unlocking position, allowing the holder to move to the activated position. Protective means are configured to prevent the locker from inadvertently damaging and passing by the first abutment surface if the autoinjector falls down onto a floor, proximal end first.

### CROSS-REFERENCE TO RELATED APPLICATIONS

This application is the United States national phase of International Application No. PCT/EP2022/066907 filed Jun. 21, 2022, and claims priority to European Patent Application No. 21305880.3 filed Jun. 25, 2021, the disclosures of which are hereby incorporated by reference in their entireties.

### BACKGROUND OF THE INVENTION

### Field of the Invention

The present invention relates to an autoinjector.

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

Automatic injection devices are designed for automatic injection of a medical product into an injection site. Autoinjectors usually comprise a housing for receiving a medical container having a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to shield or unveil the needle and an injection mechanism for automatically injecting the medical into an injection site. The injection mechanism is usually triggered by a an activation component when the needle is unveiled by the safety shield mechanism. To prevent untimely triggering of the autoinjector, for example during transport or storage, autoinjectors comprise a locker that prevents the activation component from triggering the injection mechanism unless the safety shield is properly retracted to the injection position by a user applying the autoinjector against the injection site.

There are three types of syringe flanges: the “cut flange” CF (FIG. 1A), the “round flange” and the “small round flange” RFS (FIG. 1B). The cut flange includes an anti-rolling feature in the form of two parallel edges a, b. The round flange and small round flange (FIG. 1B) have a circular shape. The small round flange has smaller dimensions than the cut flange or the round flange.

Autoinjectors are usually designed to cooperate with a cut flange. However, the cut flange may sometimes interfere with the safety shield mechanism because of its large diameter. In some cases, there is thus a need to use small round flanges, as illustrated in FIG. 1B, in autoinjectors. Small round flanges have a lower diameter than the cut flanges and thus avoid interference with the safety shield mechanism.

### DESCRIPTION OF RELATED ART

To assess the robustness of the autoinjectors, autoinjectors are subjected to drop tests as required in ISO11608. These drop tests usually consist in dropping the autoinjectors at least once from a height of 1 m onto a horizontal floor. There are three drop directions, as illustrated in FIGS. 2A to 2C: a drop ‘cap upward’ (FIG. 2A), a drop ‘cap downward’ (FIG. 2B), and a drop with the autoinjector being horizontal (FIG. 2C).

Medical containers are usually accommodated in a lower part of the housing. However, the medical containers may move a short distance in the proximal direction, i.e. inside the upper part of the housing, as it happens during a drop test ‘cap upward’ (FIG. 2A) or any accidental free fall ‘cap upward’ of the autoinjector onto the floor.

In order to stop this proximal movement of the medical container, the housing is usually provided with abutment protrusions that protrude from an inner wall of the upper part of the housing. These abutment protrusions are designed to abut against the cut flange or round flange of the medical container. This avoids that the medical container moves too far in the upper housing and accidentally triggers the injection mechanism.

However, a problem occurs when the medical container has a small round flange. Due to its reduced dimensions, the small round flange is no more capable of abutting against the abutment protrusions c of the housing (see FIGS. 3A-3B and 4A-4B). As a result, during a drop test ‘cap upward’, the medical container is free to move proximally inside the upper part of the housing. Generally, the medical container will abut against the locker and then propel the locker in the proximal direction. The medical container, filled with the medical product, heavily weighs on said locker. The kinetic energy of the filled medical container hitting the locker may cause the locker to reach an unlocking position, and may even cause the locker to move the activation component which could then trigger the injection mechanism. That is, the autoinjector may be inadvertently activated.

The document WO2019011688 discloses an administration assembly for a medicament delivery device.

There is therefore a need for an autoinjector that may be provided with a small round flange and that does not transition from a deactivated condition to activated condition when falling down on the floor during a drop test ‘cap upward’ or any accidental drop.

### SUMMARY OF THE INVENTION

An aspect of the invention is an autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism,
a locker coupled to the holder, the locker being movable with regard to the housing between a locking position, wherein the locker abuts against a first abutment surface of the housing, thereby preventing the holder from moving to the activated position, and an unlocking position, wherein the locker rotates around the longitudinal axis A and moves away from the first abutment surface, thereby allowing the holder to move to the activated position, rotation of the locker from the locking to the unlocking position being caused by the needle cover moving towards the retracted position, and
protective means configured to prevent the locker from inadvertently damaging and passing by the first abutment surface if the autoinjector falls down onto a floor, proximal end first.

Therefore, the autoinjector of the invention remains in a deactivated condition.

Thanks to the protective means, the first abutment surface resists the impact caused by the locker hitting the first abutment surface when the autoinjector hits the floor. Because of the small round flange, the medical container is not retained by the housing and therefore the locker carries the weight and kinetic energy of the medical container filled with the medical product. Since the filled medical container weighs heavily on the locker, all the kinetic energy would be received by the first abutment surface. In the absence of the protective means, the first abutment surface would be damaged and would not stop the locker, resulting in the locker moving to the uncloking position. Therefore, the protective means permit the first abutment surface to resist the impact so that the locker stays in the locking position and so that the holder cannot move to the activated position. The autoinjector remains deactivated.

The protective means may be configured to increase a contact area between the locker and the housing. They strengthen the blocking action (in an axial direction) of the first abutment surface of the housing, should the autoinjector falls down ‘cap upward’ onto the floor, but permits rotation of the locker, so that the locker may move to the unlocking position in normal use of the autoinjector.

The holder is axially movable along the longitudinal axis A between the passive and the active position. The proximal movement of the holder from the passive to the active position is caused by the needle cover abutting against the holder when the needle cover moves towards the retracted position. In the passive position, the holder may be axially away from a locking ring, and the locking ring is in a first position in which the locking ring prevents axial movement of the plunger rod. In the active position position, the holder abuts against the locking ring and has pushed the locking ring to a second position, in which the locking ring no longer prevents axial movement of the plunger rod.

The locker is axially movable along the longitudinal axis A between an initial position, in which the locker (and more specifically a proximal abutment surface of the locker) is axially away from the first abutment surface of the housing, and an intermediate blocking position (locking position), in which the locker (and more specifically its proximal abutment surface) abuts against the first abutment surface of the housing. The locker thus stands between the first abutment surface of the housing and the holder (more specifically a proximal shoulder of the holder). The housing blocks the locker and the locker blocks the holder in the proximal direction. Inadvertent triggering of the injection mechanism, such as during a drop “cap upward” is accordingly prevented.

The locker is further rotationally movable around the longitudinal axis A between the intermediate blocking position and a release position, in which the locker (and more specifically its proximal abutment surface) is circumferentially shifted away from the first abutment surface of the housing. After said rotation, the locker is free to move in the proximal direction and the holder is thus no longer prevented from proximally moving towards the active position.

In an embodiment, the protective means comprise the first abutment surface and the locker having auto-engaging shapes.

By auto-engaging shape it is meant a shape that strengthens the contact between the first abutment surface of the housing and the locker as the locker is pressed against the first abutment surface of the housing.

In an embodiment, the first abutment surface includes a slanted wall forming an acute angle with an inner wall of the housing and defining a recess configured to be engaged by the locker.

In an embodiment, the first abutment surface and the locker have complementary Z-shaped portions.

Alternatively, any shape that interlock in axial direction while allowing rotation of the locker with regard to the housing may be used as an auto-engaging shape.

In an embodiment, the first abutment surface is located on a first axial rib protruding from an inner wall of the housing.

In an embodiment, the protective means include the first abutment surface having a constant or increasing width in an inward direction.

In an embodiment, the first abutment surface has a rectangular shape.

In an embodiment, the protective means includes the locker having a radial protrusion configured to abut against a second abutment surface of the housing.

This increases the contact area between the locker and the housing and thus distributes the load on a larger surface.

In an embodiment, the second abutment surface is located on a second axial rib of the housing.

In an embodiment, the second abutment surface is flat and orthogonal to the longitudinal axis A.

In an embodiment, the locker has a ring portion and a distal leg axially protruding from the ring portion, the distal leg having a proximal abutment surface for abutting against the first abutment surface of the housing, and the radial protrusion of the locker protrudes from the ring portion of the locker.

In an embodiment, the locker has several radial protrusions and the housing has several second abutment surfaces, the protective means including a circumferential rib extending around the ring portion of the locker for connecting the several radial protrusions.

In an embodiment, the radial protrusions protrude from a top part of said rib.

In an embodiment, the axial distance between the proximal abutment surface and the radial protrusion of the locker is similar to the axial distance between the first and second abutment surfaces of the housing so that the proximal abutment surface and the radial protrusion of the locker simultaneously come in abutment against the housing.

In an embodiment, the medical container has a cut flange capable of abutting against the second abutment surface(s) of the housing.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIGS. 1A and 1B are perspective views of, respectively, a so-called cut flange and a so-called small round flange,

FIGS. 2A-2C are perspective views of, respectively, a first vertical drop position (cap upward), a second vertical drop position (cap downward), and a third drop position (horizontal),

FIGS. 3A and 3B are perspective views of autoinjectors provided with, respectively, a syringe cut flange and a syringe small round flange,

FIGS. 4A and 4B are cross-section views of autoinjectors provided with, respectively, a syringe cut flange and a syringe small round flange, in a plane orthogonal to a longitudinal axis A,

FIGS. 5A and 5B are are cross-section views of autoinjectors provided with, respectively, a syringe cut flange and a syringe small round flange, in a longitudinal plane including the longitudinal axis A,

FIG. 6 is a partial cross-section view of an autoinjector according to an embodiment of the invention,

FIG. 7 is a perspective view of the lower part and the upper part of an autoinjector according to an embodiment of the invention,

FIG. 8 is an exploded view of an autoinjector according to an embodiment of the invention,

FIG. 9 is a perspective view of a holder and a locker of an autoinjector according to an embodiment of the invention,

FIG. 10 is a perspective view of a locker of an autoinjector according to an embodiment of the invention,

FIG. 11 is a cross-section view in perspective of an upper housing of an autoinjector according to an embodiment of the invention,

FIG. 12A is a schematic view of an autoinjector according to an embodiment of the invention,

FIG. 12B is a cross-section view of an autoinjector according to an embodiment of the invention,

FIG. 13 is a cross-section view of an autoinjector according to an embodiment of the invention,

FIG. 14 is a cross-section view of an autoinjector according to an embodiment of the invention,

FIGS. 15-27 are perspective views illustrating operating steps of an autoinjector according to an embodiment of the invention.

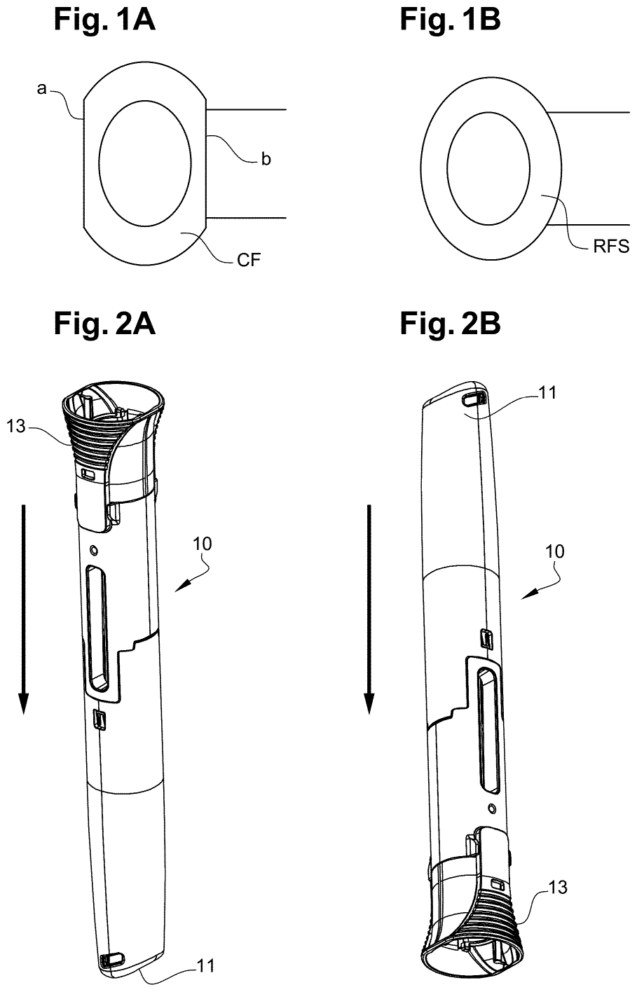

### DESCRIPTION OF THE INVENTION

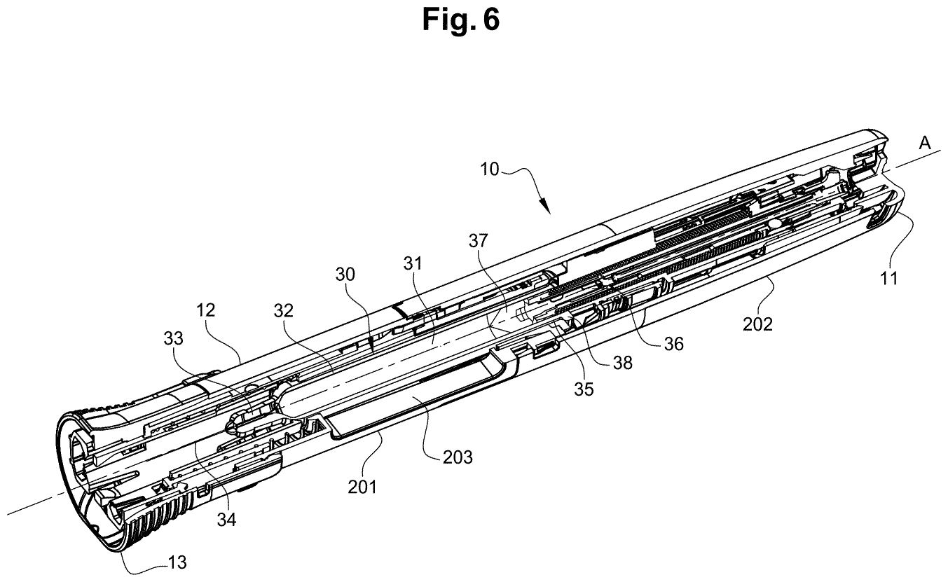

With reference to FIGS. 6 and 7 is shown an autoinjector 10 according to an embodiment of the invention. The autoinjector 10 is designed for automatic injection of a product 31 into an injection site. The autoinjector 10 comprises a housing 20 extending along a longitudinal axis A. The housing 20 is formed by the assembly of a lower housing 201 and an upper housing 202. The lower housing 201 and the upper housing 202 may be removably secured one to another by securing means such as, for instance, snap-fitting means. The autoinjector 10 has a proximal end 11 and a distal end 12. A cap 13, including a retainer 14, is removably attached to the distal end 12.

The lower housing 201 is configured to receive a medical container 30, such as a prefilled syringe. The medical container 30 has a barrel 32 defining a reservoir for containing the product 31. The lower housing 201 may define an inspection window 203 for allowing a user to visually inspect the product 31 contained within the reservoir of the medical container 30.

The barrel 32 has a distal end 33 provided with a needle 34 and an opened proximal end 35 for receiving a plunger rod 36. The plunger rod 36 may have a threaded or preferably a non-threaded distal end. The plunger rod 36 is configured to distally push a stopper 37 arranged inside the barrel 32 so as to expel the product 31 via the distal end and the needle 34. The opened proximal end 35 of the barrel 32 includes a small round flange 38 as illustrated in FIG. 6.

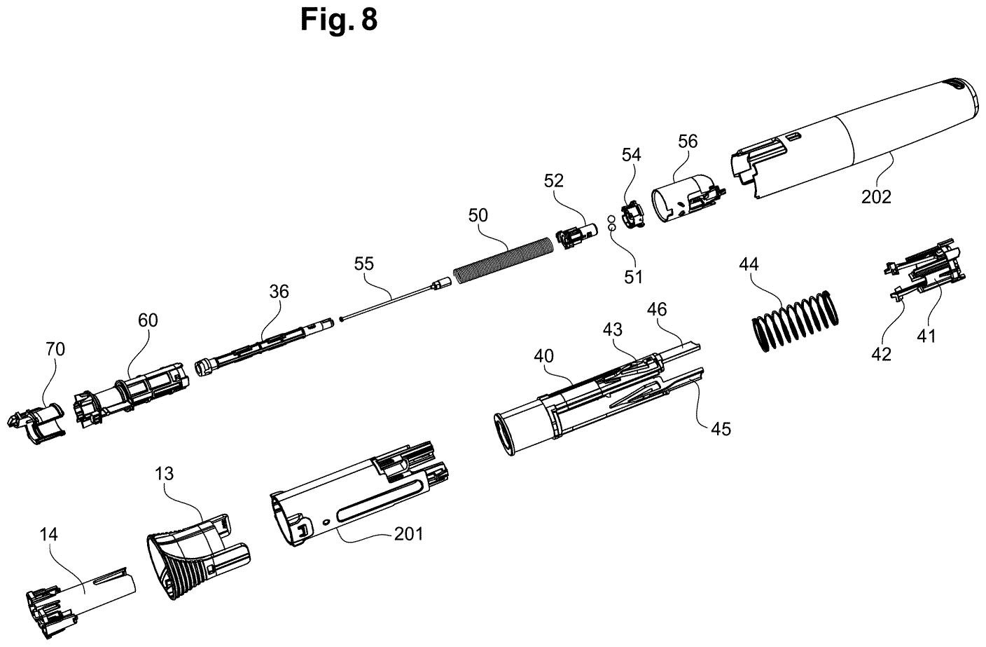

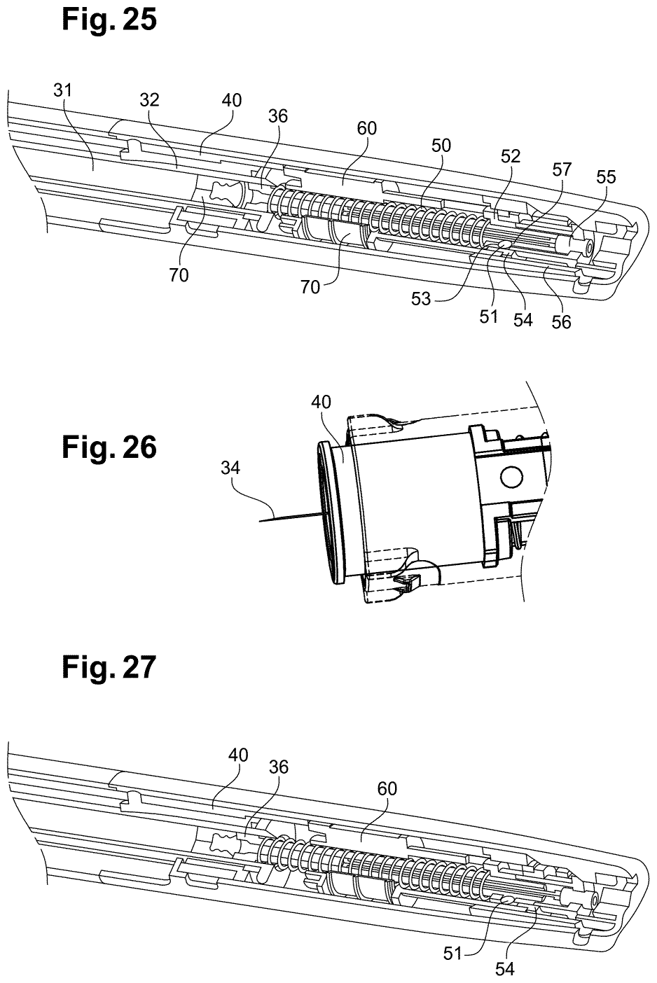

With reference to FIG. 8, the autoinjector 10 comprises a safety shield mechanism including a needle cover 40. The needle cover 40 is coupled to and movable with respect to the lower housing 201 between a first extended position (pre-use position), wherein the needle cover 40 at least partially shields the needle 34, a retracted position (injection position) wherein the needle cover 40 moves proximally inside the lower housing 201 to unshield the needle 34 (FIG. 25), thereby allowing insertion of the needle 34 into the injection site, and a second extended position (safety position), wherein the needle cover 40 moves back in the proximal direction so as to safely shield the needle 34. The safety shield mechanism includes locking means for locking the needle cover 40 in the second extended position (safety position), thereby preventing needle stick injuries. The locking means may include a locking element 41 having a proximally extending resilient leg 42 that engages a two-way slot 43 arranged through the needle cover 40. The safety shield mechanism further includes return means, such as a safety spring 44, configured to move the needle cover 40 from the retracted position to the second extended position after injection of the medical product 31 into the injection site. The needle cover 40 has a first and a second proximally extending legs 45, 46 whose function will be hereinafter described in further details.

The autoinjector 10 has an injection mechanism configured to automatically perform injection of the product 31 into the injection site. The injection mechanism includes an injection spring 50 for pushing the plunger rod 36 in the distal direction, locking balls 51 radially movable from a locking position wherein they block distal movement of the plunger rod 36 to an unlocking position wherein the locking balls 51 allow the plunger rod 36 to move in the distal direction under the action of the injection spring 50, thereby performing the injection. The injection mechanism has a washer 52 defining a radial cavity 53 for accommodating the locking balls 51, and a ring 54 coupled to and movable with respect to the washer 52 between a first position wherein the ring 54 maintains the locking balls 51 inside a recess 57 of the plunger rod 36 and a second position wherein the ring 54 allows the locking balls 51 to leave the plunger rod recess 57. The autoinjector 10 may further include a centerpiece 55 and an indicator 56 for providing the user with an audible, visual or tactile feedback that the injection is completed.

The autoinjector 10 has a holder 60 configured to trigger the injection mechanism. The holder 60 is movable within the upper housing 202 from a passive to an active position. In the passive position, the holder 60 is away from the ring 54 and thus does not trigger the injection. In the activated position, the holder 60 moves the ring 54 from the first to the second position. The proximal movement of the holder 60 from the passive to the activated position is caused by the second proximal leg 46 of the needle cover 40 abutting against the holder 60 when the needle cover 40 moves from the first extended position (pre-use position) towards the retracted position (injection position).

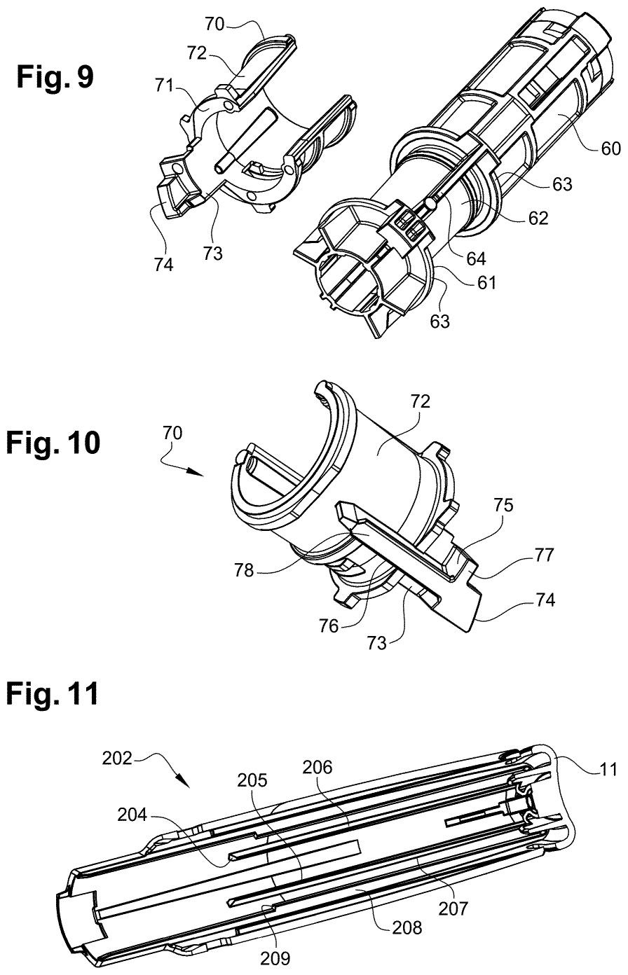

The autoinjector 10 includes a locker 70, in the form of a C-shaped ring, as illustrated in FIGS. 9-10. The locker 70 is configured to prevent inadvertent actuation of the autoinjector 10 and, more specifically, to block any accidental movement of the holder 60 towards the activated position. To that end, the locker 70 is coupled to and movable with respect to the holder 60. The locker 70 has a distal abutment surface 71 for abutting against a proximal shoulder 61 of the holder 60. The locker 70 may slide and rotate around the longitudinal axis A with respect to the holder 60 and the upper housing 202. The holder 60 has a groove 62 for accommodating the locker 70. The groove 62 may be defined by two circumferential ribs 63 that limit the axial movement of the locker 70 relative to the holder 60. The proximal shoulder 61 of the holder 60 may be delimited by one of these circumferential ribs 63. The groove 62 may further include an axial rib 64 that blocks the rotational movement of the locker 70 around the holder 60.

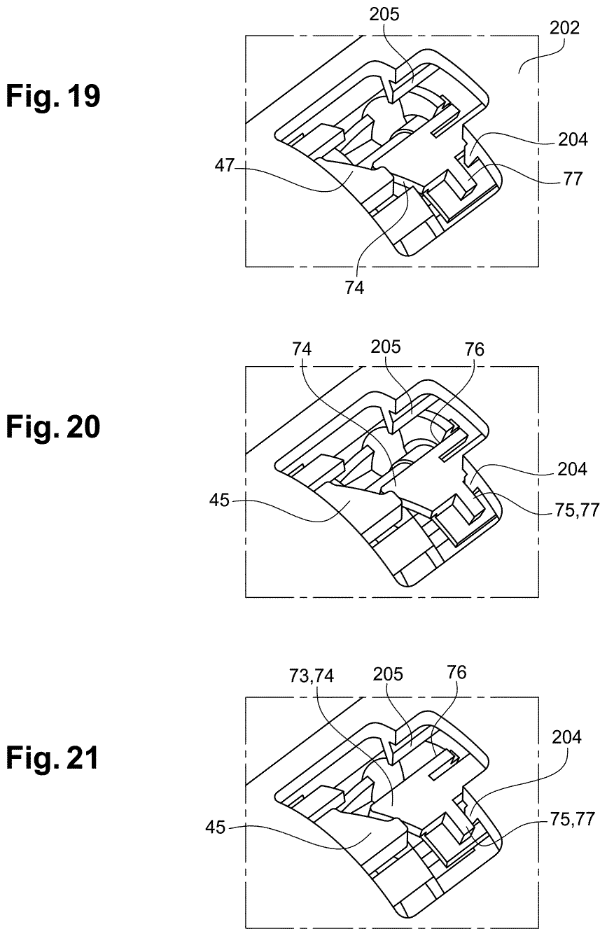

The locker 70 has a ring portion 72, that slides in the groove 62 of the holder 60, and a distal leg 73, that distally protrudes from the ring portion 72. The distal leg 73 has a cam portion 74 (at a distal end thereof), a proximal abutment surface 75 and a lateral abutment surface 76. The cam portion 74 defines a slanted wall. The proximal abutment surface 75 may be arranged on a transversal rib 77, which may extend orthogonal to the longitudinal axis A, and which is configured to abut against a first abutment surface 204 of the upper housing 202. The lateral abutment surface 76, which may be formed on a side of the cam portion 74 (FIGS. 9-10) or on a side of an axial rib 78 (FIGS. 19-22), is configured to abut against a second abutment surface 205 of the upper housing 202.

Both translation and rotation of the locker 70 are caused by the first proximal leg 45 of the needle cover 40 abutting against the cam portion 74 of the locker 70. As illustrated in FIGS. 18-22, the first proximal leg 45 of the needle cover 40 may have a cam portion 47 at its proximal end. This proximal end of the needle cover 40 and the distal end of the locker distal leg 73 may be complementarily shaped.

With reference to FIG. 11, the upper housing 202 may have one or several first axial ribs 206, one or several second axial ribs 207, and one or several third axial ribs 208, extending along the longitudinal axis A and protruding from an inner lateral wall of the upper housing 202. The first axial rib 206 defines the first abutment surface 204, which may be arranged at a distal end of this first axial rib 206. The first abutment surface 204 is configured to stop a proximal movement of the locker 70, by abutting against the proximal abutment surface 75 of the locker 70, and thus temporarily block the locker 70 in an intermediate blocking position. The second axial rib 207 has a second abutment surface 205, which may be arranged at one side of the second axial rib 207, configured to stop rotation of the locker 70, by abutting against the lateral abutment surface 76 of the locker 70, thereby positioning the locker 70 in a release position. The third axial rib 208 defines a third abutment surface 209, which may be arranged on a distal shoulder of the third axial rib 208.

The locker 70 is movable between an initial position wherein the locker 70 is away from the first abutment surface 204 of the upper housing 202, an intermediate blocking position wherein the locker 70 moves proximally and abuts against the first abutment surface 204 of the upper housing 202, and a release position wherein the locker 70 rotates away from the first axial rib 206 and abuts against the second axial rib 207 of the upper housing 202. In the intermediate blocking position, the locker 70 prevents the holder 60 from being moved towards the activated position, because the proximal shoulder 61 of the holder 60 would abut against the distal abutment surface 71 of the locker 70 and the locker 70 abuts against the first abutment surface 204 of the upper housing 202. Thus, the autoinjector 10 remains in a deactivated condition. In the release position (i.e. after rotation), the locker 70 is free to move proximally and accordingly allows the holder 60 to move proximally towards the activated position. In the release position of the locker 70, the autoinjector 10 is ready for activation. The autoinjector 10 is activated (the injection mechanism is triggered) when the holder 60 reaches the activated position.

As above-mentioned, the movement of the locker 70 from the initial to the intermediate blocking position, and then to the release position, i.e. the normal activation of the autoinjector 10, is caused by the needle cover 40 abutting against the cam portion 74 of the locker 70 while the needle cover 40 moves from the first extended position to the retracted position.

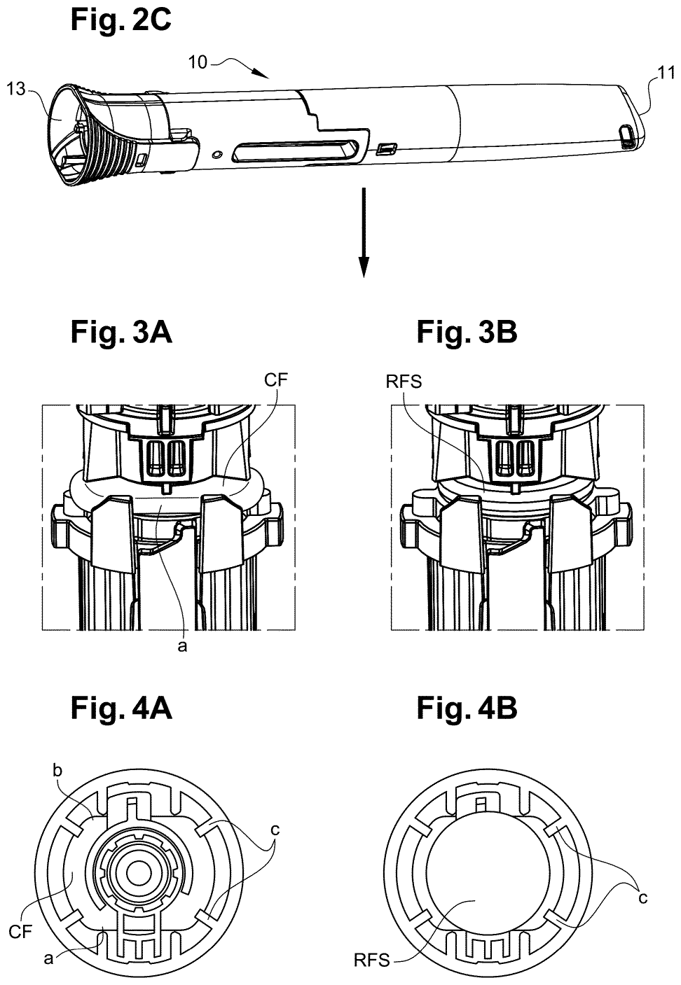

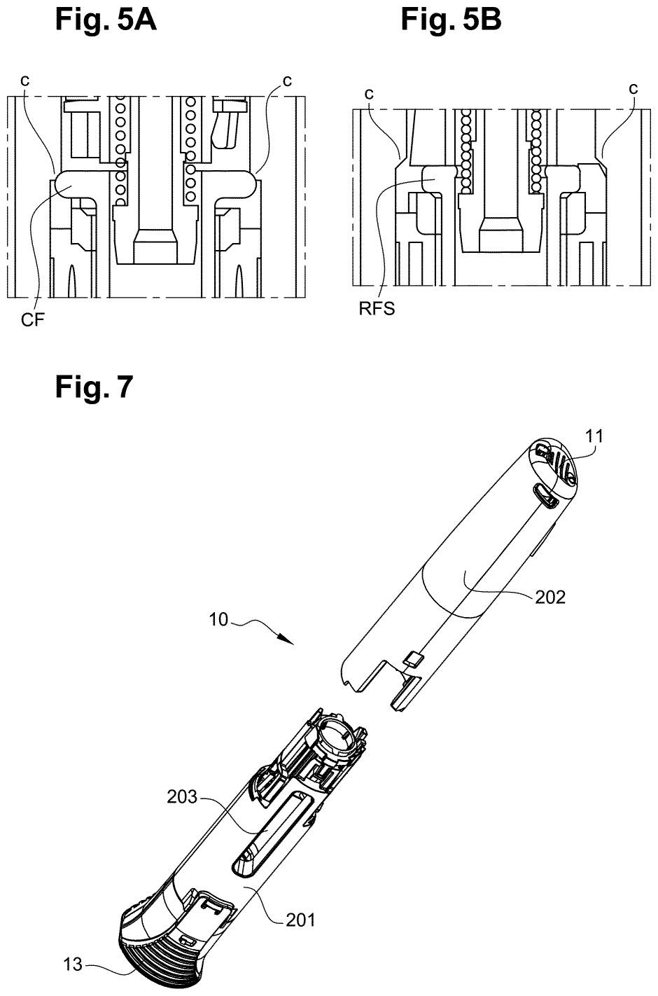

In order to prevent inadvertent transition of the autoinjector 10 from the deactivated condition to the activated condition during a drop test ‘cap upward’ or ‘proximal end first’ (as illustrated in FIG. 2A) wherein the medical container 30 having a small round flange 38 heavily weighs on the locker 70, the autoinjector 10 comprises protective means for protecting the first abutment surface 204 should the autoinjector 1 falls down ‘cap upward’ onto the floor. More specifically, the protective means may be configured to increase a contact area between the locker 70 and the upper housing 202.

The protective means are configured to prevent the locker 70 from damaging the first abutment surface 204 of the housing 20 in case the autoinjector 10 falls ‘cap upward’. Indeed, damage to the first abutment surface 204 may cause the first abutment surface 204 to let the locker 70 move together with the holder 60 towards the activated position instead of blocking the locker 70 in the intermediate blocking position (which precisely serves to prevent accidental movement of the holder 60 to the deactivated position).

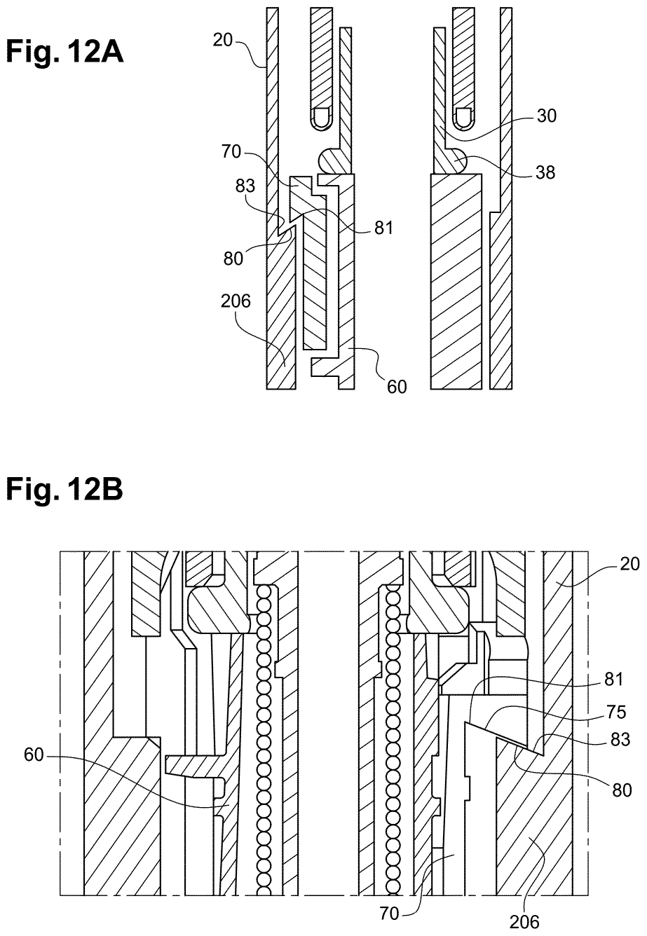

According to a first embodiment illustrated in FIGS. 12A-12B, the protective means comprise the first abutment surface 204 and/or the locker 70 having auto-engaging shapes. The auto-engaging shapes are configured to create an interlocking engagement that safely blocks axial movement of the locker 70 in the proximal direction when the locker 70 hits the first abutment surface 204, but that allows a rotation of the locker 70 around the longitudinal axis A.

For example, the first abutment surface 204 of the housing 20 may have a slanted wall 80 that defines an acute angle with the inner wall of the upper housing 202. The locker 70 may have a recess 81 for engaging the slanted wall 80, such that the locker 70 is axially trapped between the inner wall of the housing 20 and the slanted wall 80 of the first abutment surface 204. To enhance the interlocking engagement, the proximal abutment surface 75 of the locker 70 and the first abutment surface 204 of the housing 20 may be complementarily shaped, as illustrated in FIGS. 12A-12B.

The slanted wall 80 and the inner wall of the housing 20 define a circumferential slot 83 that is opened at one end or both ends so as to allow rotational disengagement of the locker 70, thereby allowing movement of the locker 70 from the intermediate blocking position to the release position in normal use.

Therefore, the auto-engaging shapes of the first axial rib 206 and the locker 70 help reinforce the contact between the locker 70 and the housing 20. Thus the locker 70 cannot by-pass the first axial rib 206 because of a drop test, even though the autoinjector 10 comprises a small round flange 38. The autoinjector 10 accordingly safely remains in the deactivated condition.

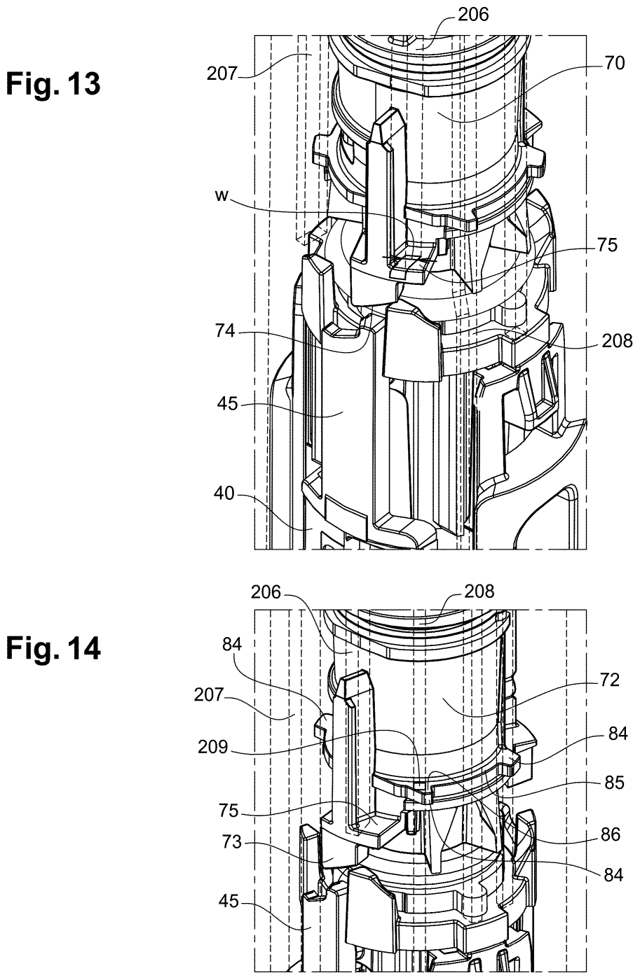

According to a another embodiment illustrated in FIGS. 13, the protective means include the first abutment surface 204 or first axial rib 206 having a radially constant, or centripetally increasing, width w. That is, the first abutment surface 204 or first axial rib 206 widens towards the central longitudinal axis A. And the top portion of the first axial rib 206 (furthest from the housing inner wall) is equal to or greater than the width of the bottom portion of the first axial rib 206 (closest to the housing inner wall).

In the example illustrated in FIG. 13, the first abutment surface 204 has a rectangular shape, and/or the first axial rib 206 has a rectangular shaped cross-section. Their width is accordingly constant in a centripetal direction. Alternatively, the first abutment surface 204 might have a trapezoidal shape, and/or the first axial rib 206 might have a trapezoidal shaped cross-section, such that their width increases in a centripetal direction. The constant or increasing width of the first axial rib 206 towards the central longitudinal axis A permits to prevent the locker 70 from getting over one side or the other of the first abutment surface or first axial rib 206 if the autoinjector 10 falls down onto the floor ‘cap upward’. Thus, the locker 70 is stopped in the intermediate blocking position and the autoinjector 10 safely remains in the deactivated condition.

According to another embodiment illustrated in FIG. 14, the protective means includes the locker 70 having one or several radial protrusions 84 configured to abut against the one or several third abutment surfaces 209 of the housing 20. The housing 20 may have as many third abutment surfaces 209 (and as many third axial ribs 208) as the number of radial protrusions 84 of the locker 70 The radial protrusions 84 may protrude from the ring portion 72 of the locker 70. They may be axially located at the same level.

In order to reinforce the contact between the locker 70 and the housing 20, and to increase the contact area in the intermediate blocking position, the radial protrusions 84 may be connected by a circumferential rib 85 that protrudes from the ring portion 72 of the locker 70. The radial protrusions 84 protrude radially outwardly from a top portion of said circumferential rib 85.

The axial distance between the proximal abutment surface 75 of the locker 70 and the proximal abutment surfaces 86 defined by the one or several radial protrusions 84 of the locker 70 may be the same as the axial distance between the first abutment surface 204 of the housing 20 and the third abutment surface 209 of the housing 20. As a result, the proximal abutment surface 75 of the locker 70 and the radial protrusions 84 of the locker 70 simultaneously hit the housing 20 when the autoinjector 10 falls down ‘proximal end first’. This permits to distribute the load so that the first abutment surface 204 of the housing 20 is not damaged by the impact. Thus, the locker 70 does not pass beyond the first abutment surface 204 in case of a drop test (and the holder 60 cannot move towards the activated position). The autoinjector 10 remains deactivated.

The third abutment surface(s) 209 of the housing 20 may preferably be flat, orthogonal to the longitudinal axis A.

It is contemplated that the third abutment surface(s) 209 of the housing 20 may comprise some or all of the features of the first abutment surface 204 of the housing 20, such as a constant or inwardly radially increasing width, in order to further reinforce the contact between the locker 70 and the upper housing 202.

It should be noted that the third axial rib 208 may be advantageously capable of abutting against a syringe cut flange should the medical container 30 be provided with a cut flange instead of a small round flange 38.

The one or several radial protrusions 84 help stop the locker 70 in the intermediate blocking position despite the weight of the medical container 30. Thus, the holder 60 is prevented from moving towards the activated position and the autoinjector 10 safely remains in the deactivated condition, even if the autoinjector 10 falls down during a drop test ‘proximal end first’.

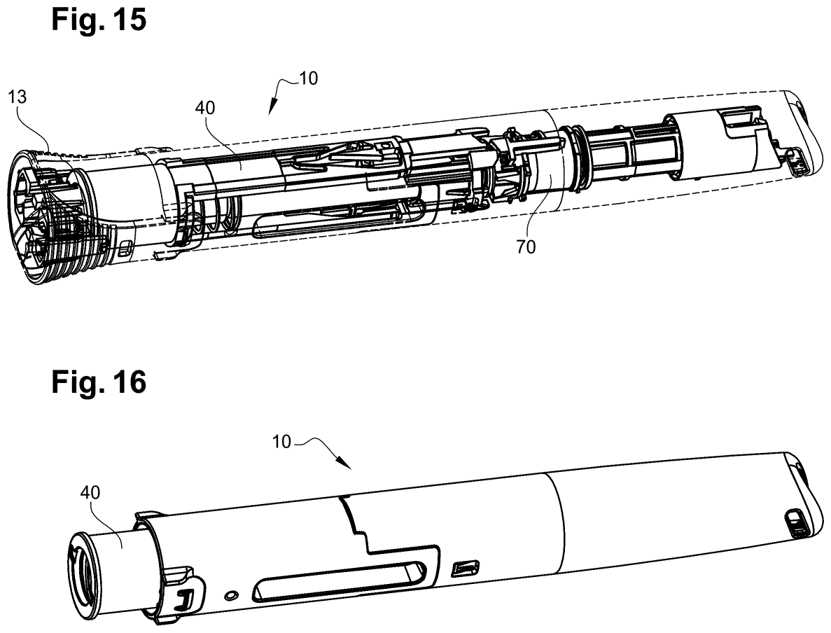

The normal operation of the autoinjector 10 will now be described hereafter with reference to the FIGS. 15-25.

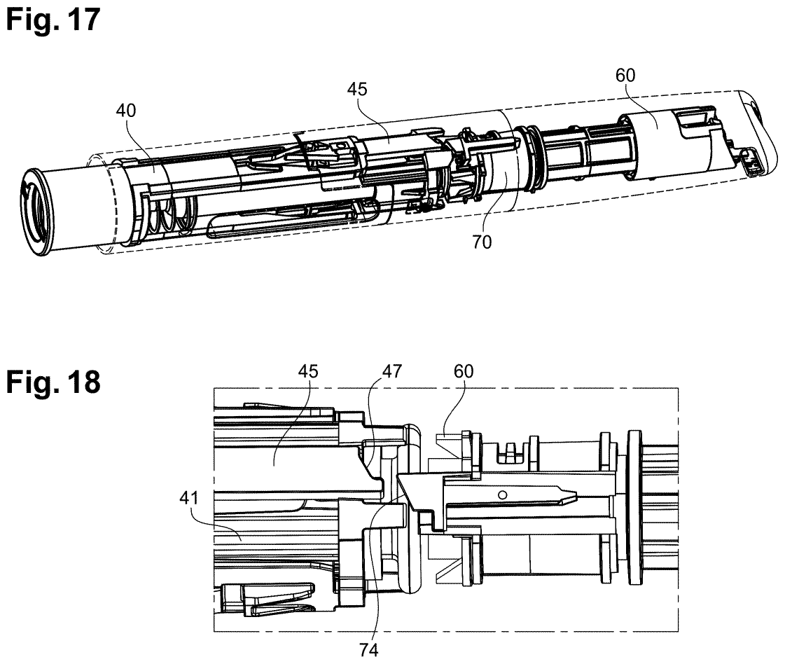

The user firstly removes the cap 13 (FIGS. 16-17). The needle cover 40 is in the first extended position (pre-use position) and therefore conceals the needle 34. The locker 70 is in the initial position and the holder 60 is in the deactivated position.

The user then applies a distal end of the needle cover 40 against the injection site and pushes the autoinjector 10 against the injection site. This causes the needle cover 40 to move in the proximal direction towards the retracted position. By doing so, the first proximal leg 45 of the needle cover 40 comes in abutment against the cam portion 74 of the locker 70 (FIGS. 17-18). Since the locker 70 is in the initial position, the proximal abutment surface is still distant from the first abutment surface 204 of the upper housing 202 (FIG. 18).

Further movement of the needle cover 40 pushes the locker 70 in the proximal direction. The locker 70 slides along the holder 60 and the housing 20 until the proximal abutment surface of the locker 70 abuts against the first abutment surface 204 of the upper housing 202 (FIG. 20). The locker 70 is in the intermediate blocking position. The locker 70 still prevents the holder 60 from reaching the activated position. The autoinjector 10 remains deactivated.

The protective means of the autoinjector 10 according to the invention help the first abutment surface 204 of the housing 20 to stop the locker 70 in this intermediate blocking position, even though the kinetic energy of the filled medical container 30 is transmitted to the first abutment surface 204 of the housing 20 when the autoinjector 10 hits the floor ‘cap upward’.

We will see below what happens next in normal use of the autoinjector 10, i.e. when a user goes on pushing the autoinjector 10 against the injection site whereas the locker 70 abuts against the first abutment surface 204.

A further movement of the needle cover 40 is required to make the autoinjector 10 transitions from the deactivated to an intermediate condition.

Due to the shape of the locker cam portion 74 and the needle cover cam portion 47, and due to the abutment between the locker 70 and the upper housing 202, further movement of the needle cover 40 in the proximal direction causes the locker 70 to rotate around the holder 60 (FIG. 21).

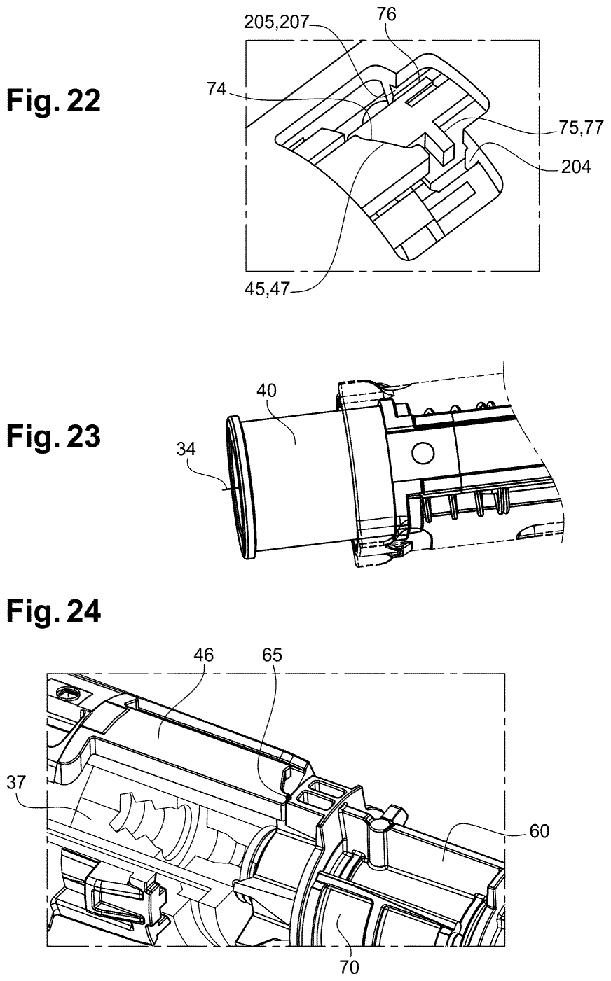

The locker 70 rotates until the lateral abutment surface comes against the second abutment surface 205 of the upper housing 202 (FIG. 22). The locker 70 is in the release position: the locker 70 no more prevents the holder 60 from moving in the proximal direction. The autoinjector 10 is ready for being activated.

At that time, the needle cover 40 may unveil the needle 34 and the needle 34 may begin insertion into the injection site (FIG. 23).

The second proximal leg 46 of the needle cover 40 comes against a distal end 65 of the holder 60 (FIG. 23) and pushes the holder 60 in the proximal direction (FIG. 24). The holder 60 leaves the passive position and progresses towards the activated position.

At this stage, the locking balls 51 extend in the locking recess of the plunger rod 36 and thus prevent the injection spring 50 from pushing the plunger rod 36 in the distal direction. The locking ring is in the first position such that the locking ring blocks the locking balls 51 inside the locking recess of the plunger rod 36.

However, when the needle cover 40 reaches the retracted position (injection position), as illustrated in FIGS. 26 and 27, the holder 60 reaches the activated position. The holder 60 comes in abutment against the locking ring and moves the locking ring in the proximal direction to the second position. The locking balls 51 are hence free to move radially outwardly, outside the locking recess of the plunger rod 36. As a result, the plunger rod 36 is no more maintained by the locking balls 51 and the injection spring 50 extends, moving the plunger rod 36 in the distal direction. The plunger rod 36 then pushes the stopper 37 inside the barrel 32, thereby expelling the product 31 through the needle 34 into the injection site. Injection is triggered, and the autoinjector 10 is activated.

At the end of the injection, the centerpiece 55 cooperates with the plunger rod 36 and the indicator to provide the user with a feedback indicating that the injection is completed.

The user may take the autoinjector 10 away from the injection site. The safety spring 44 accordingly moves the needle cover 40 back in the distal direction, until the needle cover 40 reaches the second extended position (safety position) wherein the needle cover 40 shields the needle 34 and is prevented from moving back in the retracted position by the locking element 41.

By reinforcing the contact between the locker 70 and the upper housing 202, the invention permits to prevent inadvertent activation of the autoinjector 10 during a drop test ‘proximal end first’. The protective means indeed safely prevent damage to the first abutment surface 204 (and thus allow the first abutment surface 204 of the housing 20 to reliably stop the locker 70) when the locker 70 hits the first abutment surface 204 of the housing 20, even though the medical container 30 heavily propels the locker 70 against the first abutment surface 204 of the housing 20 when the autoinjector 10 hits the floor.

1. An autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism, proximal movement of the holder from the passive to the active position being caused by the needle cover when the needle cover moves towards the retracted position,
a locker coupled to the holder, the locker being movable with regard to the housing between a locking position, in which the locker abuts against a first abutment surface of the housing, thereby preventing the holder from moving to the activated position, and an unlocking position, in which the locker has been rotated around the longitudinal axis A and moved away from the first abutment surface, thereby allowing the holder to move to the activated position, rotation of the locker from the locking to the unlocking position being caused by the needle cover moving towards the retracted position, and
protective means configured to prevent the locker from inadvertently damaging and passing by the first abutment surface if the autoinjector falls down onto a floor, proximal end first.

2. The autoinjector according to claim 1, wherein the protective means comprise the first abutment surface and the locker having auto-engaging shapes.

3. The autoinjector according to claim 2, wherein the first abutment surface includes a slanted wall forming an acute angle with an inner wall of the housing and defining a recess configured to be engaged by the locker.

4. The autoinjector according to claim 3, wherein the first abutment surface and the locker have complementary Z-shaped portions.

5. The autoinjector according to claim 1, wherein the first abutment surface is located on a first axial rib protruding from an inner wall of the housing.

6. The autoinjector according to claim 1, wherein the protective means include the first abutment surface having a constant or increasing width (w) in an inward direction.

7. The autoinjector according to claim 6, wherein the first abutment surface has a rectangular shape.

8. The autoinjector according to claim 1, wherein the protective means includes the locker having a radial protrusion configured to abut against a second abutment surface of the housing.

9. The autoinjector according to claim 8, wherein the second abutment surface is located on a second axial rib of the housing.

10. The autoinjector according to claim 8, wherein the second abutment surface is flat and orthogonal to the longitudinal axis A.

11. The autoinjector according to claim 8, wherein the locker has a ring portion and a distal leg axially protruding from the ring portion, the distal leg having a proximal abutment surface for abutting against the first abutment surface of the housing, and the radial protrusion of the locker protrudes from the ring portion of the locker.

12. The autoinjector according to claim 11, wherein the locker has several radial protrusions and the housing has several second abutment surfaces, the protective means including a circumferential rib extending around the ring portion of the locker for connecting the several radial protrusions.

13. The autoinjector according to claim 12, wherein the radial protrusions protrude from a top part of said rib.

14. The autoinjector according to claim 11, wherein the axial distance between the proximal abutment surface and the radial protrusion of the locker is similar to the axial distance between the first and second abutment surfaces of the housing so that the proximal abutment surface and the radial protrusion of the locker simultaneously come in abutment against the housing.

15. The autoinjector according to claim 8, wherein the medical container has a cut flange capable of abutting against the second abutment surface(s) of the housing.
